(19) United States
(12) Patent Application Publication
(10) Pub. No.: US 2012/0226113 A1
(43) Pub. Date: Sep. 6, 2012

(54) COMPUTERIZED INJURY MANAGEMENT PROCESS

(76) Inventor: Rajiv D. Pandya, Atlanta, GA (US)

(21) Appl. No.: 13/469,523

(22) Filed: May 11, 2012

Related U.S. Application Data
(63) Continuation-in-part of application No. 12/119,228, filed on May 12, 2008, now Pat. No. 8,219,433.

(51) Int. Cl. A61B 5/11; A61B 5/00

(52) U.S. Cl. 600/301; 600/300

(57) ABSTRACT

A method for aiding a physician in examining, diagnosing and treating an injured patient has the steps of creating a computer database that converts a selected injured body part to a sorted group of one or more preliminary diagnoses with examination protocols for each preliminary diagnosis; conducting recommended examination protocols and inputting examination results to the computer database to narrow diagnosis to most likely diagnosis; selecting a recommended diagnosis; creating a computer database of treatment protocols for every computer generated injury diagnosis, the treatment protocols having usage data and outcome data; selecting a treatment protocol; initiating treatment; and inputting treatment outcome results back into the computer database for statistical inclusion into the computer database.

### RELATED APPLICATIONS

This application is a continuation in part of U.S. application Ser. No. 12/119,228 filed on May 12, 2008 entitled “Methods For Analyzing Job Functions And Job Candidates And For Determining Their Co-Suitability”.

### FIELD OF THE INVENTION

The present invention relates to a computerized injury management process and system. More particularly, to an adaptive system that provides physicians with treatment recommendations based on the injury data inputted wherein the computer database provides a selection of diagnoses from which the physician can select from which also includes recommended treatment protocol for a particular diagnosis and more particularly a feedback data entry to constantly increase the knowledge base of the program.

### BACKGROUND OF THE INVENTION

Historically, a physician would rely on his own experience and judgment to identify what type of injury a patient had based on what he or she was seeing during an initial patient examination which would often include imaging of the injured area using x-ray or MRI scans. The severity of the injury would be determined and based on that determination; the physician would select what treatment he would give based on the options he knew and his history of success in treating similar injuries.

Another trend is to use a review of the literature to determine which treatments have shown the most promise in treating a particular injury. Repositories of outcome research, such as provided by the Cochrane Library (http://www.thecochranelibrary.com/view/0/index.html), offer an online searchable database of outcome research, categorized by injury type. Other services, such as BMJ (http://www.us.bestpractice.bmj.com/best-practice/marketing/bp-app.html) offer a subscription based service allowing the user online access to diagnosis guidelines and treatment protocols considered to be “Best Practices” for a particular diagnosis.

This access to other treatments that are available provides a resource beyond those limited to the physician's own experience and training

One of the limitations of this type of research is the physician's initial diagnosis itself may be flawed or incomplete and as a result the research may advise a treatment or repair procedure more invasive or less invasive than required due to initial misinterpretation of the injury.

Preferably, the physician could be aided in the entire procedure of evaluation diagnosis and treatment if the research tools available to him were more interactive.

In U.S. Pat. No. 7,705,727 issued Apr. 27, 2010 by Stanley L Pestotnik, et al entitled “System, Method And Computer Program For Interfacing An Expert System To A Clinical Information System” which proposed a flow of information and actions between the expert and system and clinical system and allows maintenance of audit logs in both systems. The patent allows a physician to use an expert system while specifically maintaining separate patient data. This reduces the data entry burden on the physician while maintaining patient privacy.

In U.S. Pat. No. 7,490,085 issued on Feb. 10, 2009, Matthew J. Walker, et al received a patent entitled “Computer-Assisted Data Processing System And Method Incorporating Automated Learning”. In that patent, a technique is provided for enhancing performance of computer-assisted data operating algorithms in a medical context. Datasets are compiled and accessed, which may include data from a wide range of resources, including controllable and prescribable resources, such as imaging systems. The datasets are analyzed by a human expert or medical professional, and the algorithms are modified based upon feedback from the human expert or professional. Modifications may be made to a wide range of algorithms and based upon a wide range of data, such as available from an integrated knowledge base. Modifications may be made in sub-modules of the algorithms providing enhanced functionality. Modifications may also be made on various bases, including patient-specific changes, population-specific changes, feature-specific changes, and so forth.

In U.S. Pat. No. 8,041,749 issued Oct. 18, 2011, entitled “Systems And Methods Of Managing Specification, Enforcement, Or Auditing Of Electronic Health Information Access Or use” Michael E. Beck teaches a way to allow access and user rights to health information. Methods and apparatus, including computer program products, related to managing specification, enforcement, or auditing of electronic health information use. In general, data characterizing a request to modify access rights to health information is received and the access rights are modified in accordance with the request, where the modifying includes modifying a property characterizing access rights of a relationship between a first user and second users, or an organization of the second users. The access rights may be independent of the health information and modification of access rights may be independent of a security of the health information.

In general, the medical professional is using computer aided systems to better address his patient's treatments and improve their quality of life.

The inventor of the present invention has received several patents on computer aided systems. In U.S. Pat. No. 6,865,581 Pandya, et al provides for analyzing jobs in terms of their tasks and physical requirements and a physician assessing a patient. Pandya most recently received an allowance dated Mar. 14, 2012 on U.S. application Ser. No. 12/119,228, Publication No. 2009-0281879 entitled, “Methods For Analyzing Job Functions And Job Candidates And For Determining Their Co-Suitability”; the subject matter of that application being incorporated by reference herein in its entirety.

In the context of making recommendations of content, this latest patent employed the combination of (1) breaking a job down into elemental tasks and the physical and mental requirements of each task; (2) a doctor, diagnosing the injured worker by inputting the injury into the medical diagnosing utility and the medical diagnosis utility then makes recommendations regarding the worker's ability to handle certain tasks and the doctor prescribes the computer generated recommendations as maximum allowable physical and mental requirements and movements; and (3) a risk assessment utility for parsing the computer database of past worker injuries to determine what elements of a job are more prone to causing injuries and the risk assessment utility being used to also adapt a job or job elements to compensate or reduce the risks. This application of the computer and associated software is a valuable tool to accelerate an injured worker's return to work (RTW) in a faster and safer fashion which minimizes re-injury.

A goal of the present invention is to provide the attending physician an interactive knowledge based system that starts at the examination of the injured patient and provides the physician examination, diagnosis and treatment protocols based on the doctor's input and preferably incorporates the treatment outcomes of every treated patient to provide a constantly updated knowledge base. Each entry adds to the statistical significance of the diagnostic and treatment databases to improve a “best practices” situation for the physicians or those persons responsible for treatment supervision such as physical therapists and trainers. Preferably, the computer software incorporates the latest version of ICD, International Classification of Disease, into the diagnosis protocol.

International Classification of Disease, 9^{th} edition, Clinical Modification is a standardized classification of disease, injuries, and causes of death, by etiology and anatomic localization and codified into a 6-digit number, which allows clinicians, statisticians, politicians, health planners and others to speak a common language, both US and internationally. ICD-10 CM, introduced in 2002, replaces ICD-9 CM with a final adoption deadline of Oct. 1, 2013, mandated by Health and Human Services (HHS). ICD-10 CM includes 68,000 diagnostic codes, five times more than ICD-9 CM. It expanded the code from five to seven characters, allowing alpha, except for U, in position four through seven (from ANN.NN to ANN.AAA A).

### SUMMARY OF THE INVENTION

In a first embodiment of the invention, a method for aiding a physician in examining, diagnosing and treating an injured patient has the steps of creating a computer database that converts a selected injured body part to a ICD code; conducting an initial examination of an injured patient to identify the injury location or body part or multiple injury locations or body parts and inputting those locations or body parts into the computer database to identify an ICD code or codes; creating an ICD examination diagnosis computer database to automatically convert an ICD code to a sorted group of one or more preliminary diagnoses with examination protocols for each preliminary diagnosis; conducting recommended examination protocols and inputting examination results to the computer database to narrow diagnosis to most likely diagnosis; selecting a recommended diagnosis; creating a computer database of treatment protocols for every computer generated injury diagnosis, the treatment protocols having usage data and outcome data; selecting a treatment protocol; initiating treatment; and inputting treatment outcome results back into the computer database for statistical inclusion into the computer database.

In a second embodiment, the use of the ICD code can be eliminated and can be substituted with or optionally embedded in a computer test database that provides a list of tests suggested by the injured body part identified and sorted diagnosis database that offers diagnosis based on tested results. The tests narrow the diagnosis to most likely diagnosis. Wherein the physician selects a recommended diagnosis initiates treatment and inputs treatment outcome results for statistical inclusion into the database.

A preferred embodiment is a method and computer system for executing the method for determining whether an injured player/athlete can return to play in their current position, a different existing position, the current position but altered in some form, or a different existing position but altered in some form to result in lost time reduction, having the steps of (a) creating a computer database by conducting an elemental analysis of a player's position's functions and requirements for a particular sport by breaking a position down into elemental tasks and the physical and mental requirements of each task; creating a parsable database of the positions, tasks and elements to establish specific maximum physical requirements and required movements; and storing this elemental analysis on the computer database; (b) conducting an initial medical diagnosis of the injured player by a physician examination of the player and inputting the computer database with the initial injury report to determine the physical capabilities and limitations of the player wherein the medical diagnosis has the steps of a doctor seeing the injured player patient; the doctor, inputting the data from the examination into the computer database based on the injury; the computer database automatically diagnosing the player based on the inputted data: the computer database making recommendations regarding the player's ability to handle certain tasks; the doctor reviewing the computer database recommendations and prescribing the computer database recommendations as maximum allowable restrictions on activities of the player wherein the recommendations include specific maximum allowable physical and mental requirements and movements; and the physician's office inputting the maximum allowable restriction data into the computer database; and (c) using a risk assessment utility that is used to parse the computer database of past injuries to determine what elements of a position are more prone to causing injuries, wherein said risk assessment utility is used to also adapt a position or position elements to compensate or reduce the risks; (d) comparing by computer the elemental analysis and the medical diagnosis computer generated data to determine whether the player can function in a new assignment of a particular position, wherein the particular position is either the current position, the different existing position, the current position but altered in some form, or the different existing position but altered in some form, by performing the step of comparing the injured player's inputted computer generated medical diagnosis of allowable physical and mental requirements and movements with each position's elemental analysis requirements to either allow the injured player to safely return to play by first determining if the injured player's allowable capability exceeds each possible new assignment of elemental position requirements and then using the risk assessment utility to assess the risk for each position and altered position to assign a position for the injured player that minimizes the risk of injury for the injured player based on the computer database of past injuries; or prevent the injured player from performing any position with tasks in the computer database exceeding the injured player's allowable restrictions on activities.

The method wherein (e) the computer database has information selected from the group consisting of the team's positions, the team's players, injuries to a team's players, position candidates, required skills, required education; (f) the computer database is searched by computer according to predetermined search criteria so as to result in a match between a player and a position, between a diagnosis and a position, a possibility for adapting a player or a position to fit the criteria, or to assess the risk inherent in a position; and (g) determining whether the player can do the position.

### DEFINITIONS

As used herein and in the claims:

“Patient” as used herein a patient is any person with an injury or limited functionality that is being diagnosed and treated by a physician using the computer based system of the present invention.

“Physician” is the expert medical professional who is making the initial examination and using the computerized system to arrive at a diagnosis and treatment protocol.

“Physician's Staff” are those personnel tasked to assist the physician in the treatment of a patient and whose work affects the examination, diagnosis and treatment and data entry. These include clerical, radiologist, nurses, trainers, therapists and anyone tasked to assist in treating the patient.

“Player or Athlete” as used herein a player or athlete can be any person who on an amateur basis or professional plays a sport or as a result of the activity has a range of physical requirements needed to perform the activity. By way of example these activities can include field and track, gymnastics, swimming, dance, ballet, golf, bowling, race car driving, tennis, wrestling, boxing, hunting, cycling, as well as team sports like football, basketball, hockey, soccer, baseball, volleyball, lacrosse, etc. In addition, these functional requirements are not limited to those in high school, college or professional organized sports, but further includes workers or retirees whose ability to play or practice their desired activity is dependent on their physical health. This expanded definition is particularly important to those senior citizens desirous of maintaining physical performance and functional capabilities which can degenerate with age. This is true of amateur athletes as well as professionals in any sport or activity. As for many patients, their quality of life is dependent on the ability to participate in their favorite sport or physical activity.

### BRIEF DESCRIPTION OF THE DRAWINGS

The invention will be described by way of example and with reference to the accompanying drawings in which:

FIG. 1 is a flow chart of a first embodiment of the present invention.

FIG. 2 is a flow chart of a second embodiment of the present invention.

FIG. 3 is a flow chart of a third embodiment of the present invention.

FIG. 4 is a screen shot showing that each user will have a secure login with password protection.

FIG. 5 is a screen shot of a dashboard listing each case showing the status and other case specific information.

FIG. 6 is a screen shot showing by selecting an injured player, the screen of FIG. 6 will open which shows the history and physical particulars i.e. mechanism, location, etc driven by the injured body part selected.

FIG. 7 is a screen shot showing detail for mechanism component screen provides the user with definitions and criteria.

FIG. 8 is a screen shot of an additional page for history and physical information still driven by body part. Selections on history and physical drive the next screen which narrows down diagnosis.

FIG. 9 screen shot is research that shows the data summary for the tests conducted during the physical. It is a compilation of the different research with sensitivity and specificity—how accurate the tests are in differentiating a diagnosis.

FIG. 10 screen shot is the summary of the diagnostic research as well as the testing completed by the physician during the exam.

FIG. 11 screen shot shows the treatment regimen selected based on ASMI (Andrews Sports Medicine Institute) by level of research—were they controlled studies, anecdotal, etc.

FIG. 12 screen shot is where the actual treatment is selected based on the criteria selected in the previous screen. It is diagnosis specific, and based on the scoring of outcome research.

FIG. 13 screen shot is return to play (RTP) criteria, again driven by the choices selected in the previous screens.

FIG. 14 screen shot is the reporting screen where reports can be viewed or submitted for scoring and review.

### DETAILED DESCRIPTION OF THE INVENTION

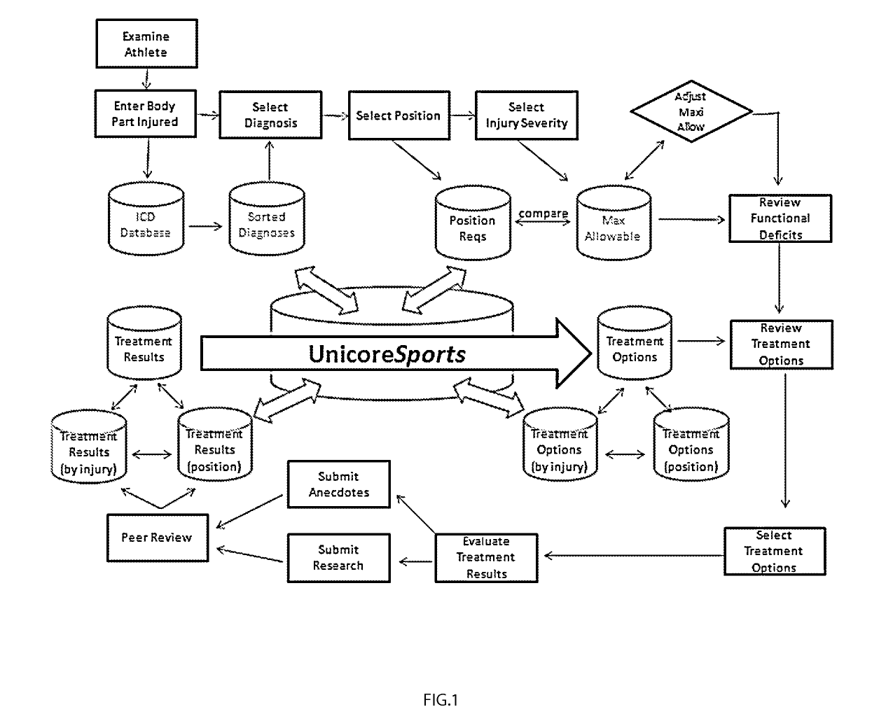

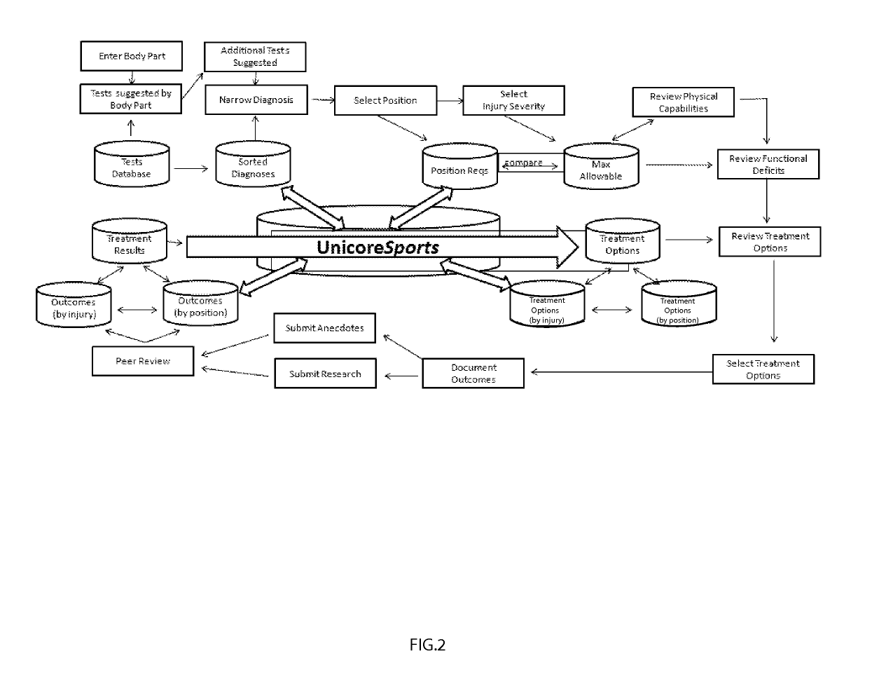

In one embodiment of the invention, using a large computer based medical program can greatly facilitate treatment of athletes. Athletes in a particular sport are required to function depending on the position played and as a result certain injuries can be more or less damaging to a players performance depending on the position played. As a result of this a computer facilitated medical diagnosis program can greatly facilitate the treatment and performance of athletes. When a player is injured, a physician can examine the athlete, identify the body part injured and enter that information into the database. The ICD database will provide a plurality of sorted diagnoses from which the user can select the desired diagnosis based on his initial examination. This initial examination can be enhanced by the computer program with preferred or recommended examination test procedures to be conducted should the physician have failed to do so or if the physician has more than one possible diagnosis that looks likely. By conducting computer recommended further examinations and testing, this diagnosis can be narrowed and optimized to increase the probability that the selected diagnosis is the most appropriate for the injury to this particular athlete. Once the diagnosis is selected, the physician needs to identify and input the player's position in the particular sport, be it basketball, soccer or football or any other sport. From the selected position there will be a computer database that has position requirements, the position requirements for a particular player's position requirements will be known. These requirements are fed into a computer database. Within the subset of requirements are the maximum allowable and possibly minimum allowable performance conditions, these are also loaded into the computer database. Once the physician knows the player's position, he can establish based on the diagnosis the injury's severity. Based on the injury's severity he can use the computer database to compare the maximum allowable conditions and adjust the maximum allowable conditions in such a fashion that he can compare the player's injury severity to the player's ability to perform. At this point, a review of functional deficits is made, once the functional deficits is established, the physician can look at the injury and the player's inability to perform at maximum allowable performance and review the functional deficits to allow a review of treatment options. In order for this to happen, the computer based program will evaluate the player based on the diagnosis and his playing position. The system then will return a list of proprietary and non-proprietary functional testing to be performed. The physician will measure the athlete's current functional capabilities and enter those into the system. The system returns a list of functional deficits and suggested treatments to remedy those deficits. Functional testing is completed at the conclusion of treatment to determine treatment success. New diagnostic, treatment and return to play outcomes are reported back to a holding database where they are peer reviewed and scored prior to release and inclusion into a master database. This entire system is shown in the flow diagram illustrated in FIG. 1. As shown in FIGS. 1A and 2, alternative embodiment flow charts are illustrated wherein the ICD code can be assimilated into and incorporated into a test database or substituted for by a more comprehensive test database or one enhanced for specialties such as orthopedic injuries or neurological injuries etc. This injury and disease management database allows for an interactive constantly updated computer program system to facilitate physicians and their ability to diagnose and treat injured people and in this particular use for players or athletes. The basis of this process involves the creation of the database which allows for input, follow-up and determination of outcomes all entered into the database. The program then searches and provides reporting as requested by the user based on a search parameter. This would include: 1) the ability to determine how many providers recommend a particular treatment and what the results of it were. 2) The development of best practices based on outcomes. 3) The initial development of suggested recommendations of treatment based on a current review of best practices, which then becomes supplemented and updated based on the learning system in the database. 4) A measure of validity and strength of recommendations is further included.

A couple of examples of such a program would be: 1) NFL database and search engine: input best practices for management of a particular injury which can be assessed by the subscriber. The provider then inputs his or her own management into the system as well as follow-ups. The system then adds this to the database, and this becomes compiled into the learning system. As the database grows, the system can deliver information that is learned to subsequent users in various formats. a) A trainer sees a player with a Grade 2 MCL sprain. He accesses the database, which provides information about the condition. The provider then adds his or her own experience. This then helps the database grow and refine the experiences for subsequent users. 2) Medical Conditions: A physician treats a patient with lung cancer using radiation. As physicians access the database to determine best practices, they enter their own experience, which helps the database grow.

An example of reporting as the living database captures information would be 9000 physicians treated sarcoidosis with high dose steroids with these outcomes. The system would also help determine the strength of the recommendations based on the number of inputs and outcomes.

The computer will integrate a series of relational databases that will allow a subscriber to use the computer to: input a diagnosis, review typical functional limitations with that diagnosis, access requirements of the athlete's playing position (return-to-play criteria), measure the deficit between those requirements and current physical capabilities, search for the best treatment to remediate that deficit, and filter, score, and report results of treatment back to the database, thereby modifying the database with those new outcome results.

Using a series of relational databases integrated into the exemplary UnicoreSports platform, subscribers are able to enter predetermined criteria into the computer, and the computer will match those criteria with relevant guidelines for diagnosis, treatment, and return to play criteria. The computer sorts and filters any new guidelines and adjusts the database accordingly. Subscribers are able to use the computer program to review, choose, and evaluate treatment practices based on an athlete's or player's injury and position on the field of play. The attached flow charts of FIGS. 1, 2 and 3 depict the process as it is currently envisioned.

One embodiment of the present invention can comprise conducting an elemental analysis of a position's functions and requirements, conducting a medical diagnosis of a player to determine the physical limitations or physical abilities the player is capable of, and comparing the elemental analysis and the medical diagnosis to determine whether the player can function in a particular position. For example, a position can be divided up into individual tasks, with each task being broken down into elemental parts. Such elemental parts can be objectively described by the physical and/or mental functions associated with each task, such as for example physical movements, weights, and repetitions involved. These elemental parts can be collected into an integrated parsable database and computerized for ease and speed of searching and comparing. In this same example, a player can undergo a medical diagnosis (a medical check-up) to determine what the player is capable of For example, in conducting a medical diagnosis of an injured player, a doctor can recommend that the player limit his activities and actions to certain maximum allowable units, such as maximum weight lifted, maximum repetitions per hour, maximum time standing or sitting, and maximum body motions such as twisting or bending. These maximum allowables also can be collected into an integrated parsable database and computerized for ease and speed of searching and comparing. The maximum allowables determined in the medical diagnosis that can be compared to the elemental analysis to determine a fit between a player and a position.

Another embodiment of the present invention can compare the elemental analysis of a database of positions to the medical diagnosis of an injured player to determine whether the player can return to play in a particular position, such as the player's pre-injury position, or in any position within a team or play location. In this embodiment, the maximum allowables resulting from the medical diagnosis initially can be compared with the elemental analysis of the player's original position to determine whether the player can return to play at his or her original position. If not, the player's maximum allowables can be compared to the elemental analyses of a portion or of all of the positions within a team or group of teams to determine whether the player can return to play in any position within the team or group of teams. This could be beneficial to organizations and players organizations alike.

A further more general embodiment of the present invention can comprise use of the elemental analysis to provide for specific needs of a team. In this embodiment, the maximum allowables can be compared to the elemental analyses of various positions and if no appropriate positions are available, or if a desired position is not appropriate, the elemental analyses can be reviewed to determine if the position can be altered to accommodate the players available. This is a valuable tool in establishing team needs in drafting or acquiring new players.

Additionally, another general embodiment of the present invention can comprise use of the elemental analysis and the specifics of injuries reported by players to create a clinically relevant risk assessment for determining the specific risks of a position, and use of the risk assessment for modifying a position's functions and requirements to suit the player or other players. For example, the various injuries occurring in connection with a specific position can be tagged to the elemental analysis. The various injuries can be assessed using standard statistical techniques to result in a determination of the likelihood of such injuries occurring in the future by players on this position. This type of determining could identify injury trends very early on and help teams and organizations to adjust rules and implement proactive measures to lower risk. Concussions and head and neck injuries in NFL football and NHL hockey are examples of problems that could easily have been discovered and addressed much earlier with the present invention. Such uncovering of these issues could have saved players and avoided litigation against the organizations.

The elemental analysis, in summary, comprises breaking a position down into its component tasks. Additionally, the specific position tasks also can be further broken down into whether the task involves repetitions tasks, and into how the tasks can be accomplished. The elemental task information is entered into a database, preferably a parsable database, and more preferably a computerized parsable database.

The medical diagnosis, in summary, comprises diagnosing a player. A typical medical or clinical diagnosis can include, for example but not limited to, providing descriptions of the player's injury, providing limitations on physical and/or mental activities, and providing a timeline for recovering from such injury and limitations. The physical and/or mental activities can be broken down into specific activities and the limitations attached to such activities. For example, but not limited to, the medical diagnosis can provide that a player can lift no more than a prescribed amount and can only twist the torso no more than 90 degrees. In other words, the medical diagnosis can provide maximum allowable actions in weight and movement. The maximum allowables can be entered into the same database as the elemental analysis, but preferably either is entered into a separate database or is maintained as separate data pertaining to a particular player. For example, while the elemental analysis data can be pertinent to all medical diagnoses and therefore preferably should be parsable relative to all players, the medical diagnoses can be and generally are specific to each individual player and their respective positions.

The comparison between the elemental analysis and the medical diagnosis, in summary, can be initiated after converting the medical diagnosis into the maximum allowables and then comparing the maximum allowables to the elemental analysis to determine whether the player's limitations or functional deficits will allow the player to do the position, whether the position has acceptable task criteria to accommodate a player, and whether the task criteria can be altered to accommodate an injured player. Medical and clinical criteria are used when creating the restrictions and maximum allowable actions from the medical diagnosis. In other words, the maximum allowable actions link the elemental analysis with the medical diagnosis.

The present invention comprises several utilities that can be carried out individually or in various combinations, or all together, to increase the chance of successfully determining whether a player, such as an injured player, can return to a previous position, or more generally matching a player to a position, and vice versa. The present invention preferably comprises (a) an elemental analysis utility for creating and using an integrated database of positions, position functions, position tasks, and position requirements; (b) a medical diagnosis utility for creating and using an integrated database of player injuries, and of injuries to a particular player; and (c) a return to play utility for more effectively allowing an injured or disabled player with functional deficits to return to play and to match position candidates with positions by comparing the medical diagnosis with the elemental analysis. Additional utilities can include (d) a risk assessment utility for parsing the integrated databases to determine the risk of a player being injured or re-injured when performing at a specific position, and (e) a modification utility for allowing the modification of a position to suit a particular player, preferably based on a comparison of the medical diagnosis with the elemental analysis. Amazingly, almost all of this work is done by teams manually without any organized way to measure a player's ability after injury or aging to move to an alternate position gaining additional productive play.

Each utility can have the ability to query the elemental analysis database to provide information relevant to the utility so as to be able, for example (a) to pair a player with the most suitable position or a position with the most suitable player, (b) to allow the adaptation of a position to a player or to address recurring injuries resulting from the position, (c) to allow the modification of a player's tasks in performing a position to address an injury or recurring injuries resulting from the position, (d) to determine whether a position candidate is suitable for a particular or any position and vice versa, (e) to use a medical or physical diagnosis of a player to determine whether a position is available for the player or whether a position can be adapted to such a player, and (f) to assess the risk of future injuries resulting from a position.

Throughout this specification, various terms will be used in a general sense and are meant to encompass or include a range of subsets. The term player includes players, disabled players, injured players, and position candidates, depending on the situation. The term team includes all types of teams at schools, universities, amateur or professional. The term position includes all positions, professional, paraprofessional, amateur, skilled or unskilled. The terms tasks or elements include the various specific activities and actions that make up a position. Although the methods and systems of this invention can be used by many different types of teams, for ease of this disclosure, the invention will be disclosed in conjunction with teams having multiple player positions.

### II. General Features

The invention can be used to implement a team-wide, play location-specific method and system for analyzing players and positions for determining, for example, whether an injured player can return to the player's position, whether a particular player is suitable for a particular position, and vice versa, and how a particular player can alter his or her play mechanics or how a particular position can be adapted to a particular player. In short, an integrated parsable database comprising elemental analysis information on the team's positions, the team's players, injuries to a team's players, position candidates, required skills, required education, and combinations of these, is created. This database is parsed according to predetermined search criteria based on the utilities disclosed above so as to result in a match between a player and a position, between a diagnosis and a position, a possibility for adapting a player or a position to fit the criteria, and/or to assess the risk inherent in a position.

A feature of the invention is the creation of a standard, objective parsable database of at least the requirements for specific positions within a specific team or organization of teams. Additional information can be included in the database such as, but not limited to, on-the-position injuries to players and how these injuries occurred, and establishing regulations and requirements for players and positions. Another feature of the invention is a means for parsing the database so as to allow the comparison of a player to a position, and vice versa, so as to determine the co-suitability of the player to the position, and vice versa. This parsing function allows the user, such as the team, to determine the best position for a player, whether a position can be adapted to a player, and the risks associated with a position relative to a player. In a simple form, the present invention can provide the team, as well as physicians, other health practitioners, trainers, coaches, human resource persons, and risk management persons, the knowledge of the tasks, elements of tasks and qualifications required of specific positions within a team, thus allowing a more objective determination of whether a person is suitable for a position, such as whether a potential player is capable of performing a specific position, whether a position can be modified for a potential or existing player, and/or whether an player can return to play, either in the original position or, if not, in another position within the team or in related or other teams.

The database can be created in many ways by inputting the desired information. For example, a task-specific position analysis can be conducted of the physical requirements for each position. For another example, a physical demands analysis can be completed giving a written and/or a pictorial description of the various functions involved in carrying out the position. In the position analysis and physical demands analysis, the various restrictions and maximum allowable physical requirements of a player, through the medical diagnosis, is compared with the restrictions or maximum allowable actions and requirements of each specific task of a position, can be quantified and included in the database. In other words, an elemental analysis of each position is conducted and the elemental restrictions and requirements are included in the database. Then the database can be parsed in connection with the various utilities of the invention.

Additionally, regulations and rules can be inputted and cross-referenced to specific positions; educational, certification, and credentialing analyses if implemented by the organization can be completed for each position; and playplace injuries, how the injuries occurred, and what effect the injuries had on the player can be compiled. All of this information can be inputted into the database, cross-referenced, and made available in a parsable format by one of ordinary skill in the database creation field. By parsing this type of database, the suitability of a player for a position or a position for a player, the risk assessment of a position or a position task, and the ergonomics of a position can be determined for an injured player in a return to play situation or for the acquiring of a new player be it through a draft or trade.

In an alternative embodiment, the present invention can help teams and players find suitable matches between the player and the team's position bank. Use of the parsable database can find alternative positions that a player can do. For example, in use, the database can be parsed by maximum weight the player is able to lift, maximum frequencies of motion or movement a player is able to do, and/or the physical activities the player is able to do. Similarly, the database can be parsed in the contracting process by allowing the player to input his or her physical limitations and positions that the team has available then will be returned, and the player and the team can decide if the player is right for the position. This is advantageous in both the return to play and selection processes.

In another alternative embodiment, the present invention can help teams use medical and physical diagnoses of players to find suitable positions for players and to adapt current positions to particular players or a particular organization. For an example in use, a medical diagnosis, which could include tasks the player can and cannot perform, can be compared with the team's positions. Positions that the team has available that satisfy the diagnosis criteria then will be returned, and the player and the team can decide if the player is right for the position. This also is advantageous in both the return to play and selection processes.

In another alternative embodiment, the present invention can help teams use player injury data to adapt positions and to assess the risk of future injuries by players carrying out positions. When injury data is cross-referenced to positions, it can more easily be determined whether a position has a higher risk of injury, and what that injury might be. Additionally, this injury information can be used to adapt the position so as to possibly reduce or eliminate the risk in the future. This is advantageous in the return to play and rules creation and adaptation processes.

A. Elemental Analysis Utility.

As already disclosed, position elemental analysis includes breaking a position down into elemental tasks and the physical and mental requirements of each task. A database of the positions and elemental tasks is created for parsing in other utilities.

B. Medical or Clinical Diagnosis.

As already disclosed, diagnosis includes using a medical or physical diagnosis of a player to determine the suitability of a player for the player's original position, a different existing position, the same position but altered in some form, or a different existing position but altered in some form, or whether a position candidate can satisfy the criteria for existing positions or for existing positions altered in some form. For example, the post-injury player may have different physical abilities than the pre-injury player, and one player may have different physical abilities and needs than another player. The medical or physical diagnosis utility can be used to determine whether a player can return to play in the same position, a different existing position, the same position but altered in some form, or a different existing position but altered in some form, or whether a position candidate can satisfy the criteria for existing positions or for existing positions altered in some form. This utility can include:

(1) Utilizing a medical or physical diagnosis that leads to restriction of a player to match that player with a particular or any position within a team;

(2) Utilizing a medical or physical diagnosis of a person to determine the restrictions for a player to take a position, based on the maximum allowable tasks for the position;

(3) Utilizing this diagnosis-based assessment of a person to translate the restrictions for a player into the maximum allowable tasks for the player so as to be able to match the player to a position and vice versa;

(4) Allowing searching of the database for a listing of possibly appropriate positions for the player, determining why or why not a position can or cannot be done by the player, and then matching the restrictions with all position elements in a team; and/or

(5) Applying the diagnosis-based assessment to qualifications or essential functions required to allow searching of the database for a listing of possibly appropriate positions for the player.

In a simple playing example, the medical diagnosis can operate as follows. The doctor sees patient, namely, the player. The doctor, based on the injury, makes a diagnosis of the player using his examination and the computer database. As part of this diagnosis, the doctor further makes recommendations regarding the player's ability to handle certain tasks. For example, as already disclosed, the recommendations may include that the player cannot do certain tasks at all for two weeks, that the player cannot lift more than a certain amount, that the player cannot twist more than 90 degrees, that the player can perform no more than a prescribed amount of repetitions per minute, et cetera. The doctor then prescribes these restrictions as the maximum allowables. Alternatively, the system, based on historical data, can use known artificial intelligence methods to prescribe restrictions.

C. Return to Play Utility.

This utility can be defined as determining whether a player can return to play in the same position, a different existing position, the same position but altered in some form, or a different existing position but altered in some form. The medical diagnosis for a particular player can be used to parse the elemental analysis database for making this determination. Similarly, this utility can be used in the acquisition or hiring of new players. That is, a new player's physical and educational skills and training can be used to determine whether the new player satisfies the criteria for existing positions or for existing positions altered in some form. The return to play utility can be used in determining whether a player can return to play in the same position, a different existing position, the same position but altered in some form, or a different existing position but altered in some form. This utility can include:

(1) More effectively allowing a player to return to play in a particular position or any position within a team;

(2) Allowing training and coaching personnel and advisors to contemporaneously communicate to determine whether a particular player can function in a particular position or in a different position within a team;

(3) More effectively getting an injured player back to play;

(4) Determining whether a player has the appropriate faculties for a specific position or for any position in the database both during the pre-employment process and post injury; and/or

(5) Allowing a ranking of positions suitable for the physical capabilities of a player and basing the ranking on positions with the most suitable elements and/or positions within or proximal to the player's pre-injury position. The graphic representation of this ranking accomplishes another important objective in that it translates the clinical terms used by the physician into the terms of the specific tasks and elements used by the team.

This utility also can comprise an acquiring or hiring utility that can be used in determining whether a player's physical and educational skills and training satisfy the criteria for existing positions or for existing positions altered in some form. This utility can include:

(1) Determining whether a player has the abilities and faculties necessary for a particular or any position within a team;

(2) Determining whether a particular position within a team is suitable for a particular or any player;

(3) Increasing the ability for a team to retain and place players in positions within the team;

(4) Creating and using a database listing maximum allowable tasks for positions, including searching the database to find a position suitable for an individual;

(5) Determining whether a player has the appropriate faculties for a specific position or for any position in the database both during the pre-hiring process and post injury;

(6) Allow a ranking of positions suitable for the physical capabilities of a player and basing the ranking on positions with the most suitable elements and/or positions within or proximal to the player's pre-injury position; and/or

(7) Allowing a matching of a player's certifications, training, and/or credentialing with the positions in a database.

D. Risk Assessment Utility.

This utility can be used to determine what elements of a position are more prone to causing injuries and to adapting positions or position elements to compensate or reduce such risks. Similar to a position elemental analysis, an elemental risk assessment can be made of each position by taking past injuries that occurred for the position and creating a database of such injuries. The risk assessment utility can be used to parse a database of past injuries to determine what elements of a position are more prone to causing injuries and to adapting positions or position elements to compensate or reduce such risks. This utility can include:

(1) Utilizing a physical demands analysis (elemental analysis) and scalability to create and use a database to help show which task of a position is associated with a specific risk of injury;

(2) Allowing a comparison of position elements for creating a risk assessment of whether a specific position or a specific task will or does have a higher risk of causing injury to a player or causing a repeat injury to a previously player; and/or

(3) Allowing the creation of risk management reports based on position tasks.

E. Modification Utility.

This utility can be used to create and modify positions based on player abilities, skills, and training For example, the player diagnosis and the risk assessment can be used to create the criteria, tasks, and elements for new positions or to adapt the criteria, tasks, and elements of existing positions to more fully employ players. The position creation and adaptation utility allows the use of player abilities, skills, and training to create the criteria, tasks, and elements for new positions or to adapt the criteria, tasks, and elements of existing positions to more fully employ players. This utility can include:

(1) Using a database of playplace injuries or other player information to assist in determining the necessary criteria for a position;

(2) Allowing a searching of positions at other related teams to allow players at one related team to be transferred to other related teams, or to allow players at one lower division or farm league team to move up to a parent team or vice versa; and/or

(3) Allowing a categorization of the positions based on various hierarchies, such as department, facility, physical requirements, essential or non-essential functions, et cetera.

### III. Database

The database can include information about positions, players, injuries, regulations, rules, et cetera. Generally, the database preferably comprises the elemental tasks of each position and allows a parsing of the database based on criteria for allowing a player to perform the tasks of a position. In this manner, players can be matched to positions that the players can physically handle. Specifically, the innovation of creating the database, matching the database up with clinical data of physical restrictions and requirements, and applying the database to players to determine player suitability for a specific position is a preferred feature of this invention. The innovation of breaking a position down into playstation, tasks and elements, matching up injury data with each playstation, task and element, and determining whether an individual playstation, task or element is more likely to cause an injury or whether an injury is more likely to occur when performing an individual playstation, task or element is another feature of this invention.

Positions are analyzed in terms of elements and tasks. An element is the smallest step into which it is practical to subdivide any play activity without analyzing separate motions, movements and mental processes involved. A task is one or more elements and is one of the distinct activities that constitute logical and necessary steps in the performance of play by the player. Further, positions may be broken down in terms of positions. A position is a collection of tasks constituting the total play assignment of a single player. Finally, team positions are a group of positions within a team setting, which are identical with respect to their major or significant tasks and sufficiently alike to justify their being covered by a single analysis.

Each element is analyzed in terms of its physical demands. Strength requirements are obtained using standard position analysis equipment. Definitions for physical requirements are taken from, for example, commonly available sources such as The Revised Handbook for Analyzing Positions published by the United States Department of Labor, Employment and Training Administration in 1991. For example, play can be categorized as sedentary play, light play, medium play, heavy play, and very heavy play. Elements can include such activities as running, standing, walking, sitting, lifting, carrying, pushing, pulling, climbing, balancing, stooping, jumping, kneeling, crouching, crawling, and reaching.

Essential functions are any element of the task that must be completed by the player without assistance and without modification. If a player is unable to perform an essential function he is unable to complete his required position duties. Thus, for example, when creating the position portion of the database, one must take into account whether removing the function fundamentally changes the position and if the function is critical to overall performance of the position, as well as other questions.

The parsable database is created to allow for more effective return to play and retention of positions. By breaking a position down into playstations, specific tasks, and inputting this elemental analysis (for example, physical position restrictions and requirements) into the parsable database, a position can be matched up a player according to essential and non-essential functions as determined by the team and weighted by the team. Similarly, a medical professional can provide clinical restrictions (maximum allowables) for a position (for example, physical restrictions based on a hypothetical ordinary or average player could or should be able to accomplish), which can be inputted into the parsable database. In this manner, the various restrictions and requirements of a position are available for parsing and matching to a player.

### IV. Parsing

The present invention has been specifically and uniquely designed to address and expedite early and appropriate return to play options. The primary component of this system is a parsable database that allows the user to view specific tasks associated with each position, including all physical demands of the position. The invention can graphically show (1) a list of the specific elements of the player's position that fall inside and outside of the parameters of the restrictions, (2) a list of other positions available with the team that are within the restrictions or are closest to the restrictions, and (3) specific injuries that occur during individual position tasks. By identifying the specific elements with their physical requirements, the user can isolate the elements that the player is unable to perform and assist in identifying alternative positions, tasks or methods that are within the imposed physical restrictions, and create a risk assessment for a particular position or position task. This accomplishes another important objective in that it translates the clinical terms used by the physician into the terms of the specific tasks and elements used by the team.

The present invention enables the teams to evaluate return-to-play issues from the beginning of care. Using the present invention, the team is able to make a more accurate disposition of the player's case, hopefully resulting in an early and safe return to play in the same position, the same position adapted based on the injury or diagnosis, a different position, or a different adapted position. Further, the team can utilize the search engine to determine if there are any other positions on the team that the player can currently perform within the restrictions set out by the physician or in the medical diagnosis. This allows a determination to be made to see if these tasks can be reasonably modified to allow the player to return to his original, albeit, modified position.

When examining the player and developing a medical diagnosis, the physician can view still photos, video and metrics regarding the player's position and thus more accurately determine the restrictions or allowable actions. The present invention will compare these restrictions with the information player's position and other positions in the database, and the system will provide a listing of positions in descending order of positions (if any) that satisfy the physician's criteria, to positions that satisfy most of the physician's criteria, to positions that satisfy only some of the physician's criteria, and so on. The team is now able to make a reasonable recommendation for the player's ability to perform the player's position and, if not, whether there are other positions in the team that the player can do in the injured and recovered state. Alternatively, the team can review the listing of positions and determine whether any of the positions can be modified to accommodate the player.

Contemporaneously, the injury data is attached in the database to the specific position and position task during which the injury occurred. As the injury data is compiled, that is when additional injury data is inputted into the database, an injury profile is created for a specific position and position task. Using this data, a risk assessment for the position and position task can be created, and the occurrence of a future injury can be predicted. Additionally, the injury data can be used to modify the position. Thus, the database can be parsed both for determining whether a current position and position task has a risk of injury or a high predictability of an injury occurring, and for positions and position tasks that could or should be modify to reduce the risk of future injury. Thus, the present invention provides a clinical model of the risk of injury due to a position or position task, what the injury is likely to be, and why the injury occurred.

A parsing of the database can generate a listing of the types of injuries occurring and the frequency of the injuries in elemental detail, that is, which position element is prone to injury. Thus, the position element can be modified (e.g., lowering a maximum lifting weight), or a different player or assistant can be assigned to the position (e.g., a stronger player to carry out the specific task), or in the case of return to play assigning the injured player to a different position. Thus, a risk prediction database can be developed for each position task, each position, or each team. This results in a very powerful tool for predicting risk based on position tasks, namely, the ability to identify which players with particular medical diagnoses can or cannot perform a position or a position task. If the position task is an essential element of the position, the diagnosed player can be assigned to a different position, the position can be modified, or an assistant can be assigned to carry out the position task.

One aspect of the parsing ability is predicting risk based on previously reported and inputted position injuries. The present invention comprises a very powerful tool for predicting injury risk because each injury is attached to a specific position task and therefore the injury risk assessment is based on elemental analysis. The database can be parsed for a particular type of restriction or a particular type of injury and, based on the injury, the invention can identify positions for players who have medical restrictions either from previous lifting injuries or a medical diagnosis. With the present invention, a team can parse players with a particular diagnosis and break down how many elemental (or position task) injuries occurred and what the elemental injuries were, and use the position elemental analysis to determine and evaluate player risk relative to a position or position task. In other words, the present invention matches a position elemental analysis with a player diagnosis to allow the team to determine whether the player is suitable for the position and vice versa. Comparing a position at the elemental analysis level with a player medical diagnosis can allow the team to determine which positions or position task is an injury risk. Thus, by capturing a medical diagnosis (injury) associated with a particular position task element and capturing that data, a team is able to determine or predict, based on particular position task elements and historical injuries throughout the entire team or entire organization, the risk of injury.

### V. Illustrative Embodiment

Referring now to the figures, an illustrative embodiment of various features of the invention is disclosed. Some of the figures are flowcharts illustrating the information gathered for input into the parsable database and how the database can be used to carry out the various utilities of the invention. Some of the figures are screen shots of a software application developed for carrying out the invention illustrating the information that can be parsed in the database and typical results that are returned. Overall, a disclosure of how the inventive database is created, updated, and used is provided by these figures and the connected disclosure.

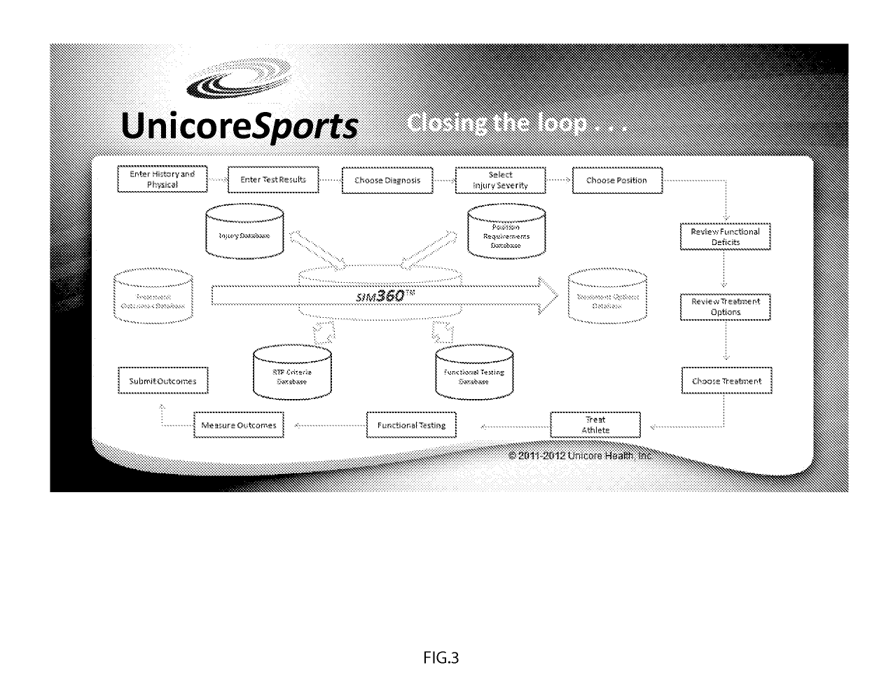

FIG. 1 is a flow chart using the computer based system wherein based on an examination, the body part injured is fed into the ICD database which provides a code from which a database of sorted diagnoses is fed back to the examining physician a number of possible diagnoses which are pared down by prompting additional tests until a recommended diagnosis or a group of ranked diagnoses are provided from which the physician arrives at a selected diagnosis. As shown in the flow charts of FIG. 2 or 3, the flow chart of FIG. 1 can be modified simply to have a test or injury database in the place of the ICD information or embedded with the ICD information.

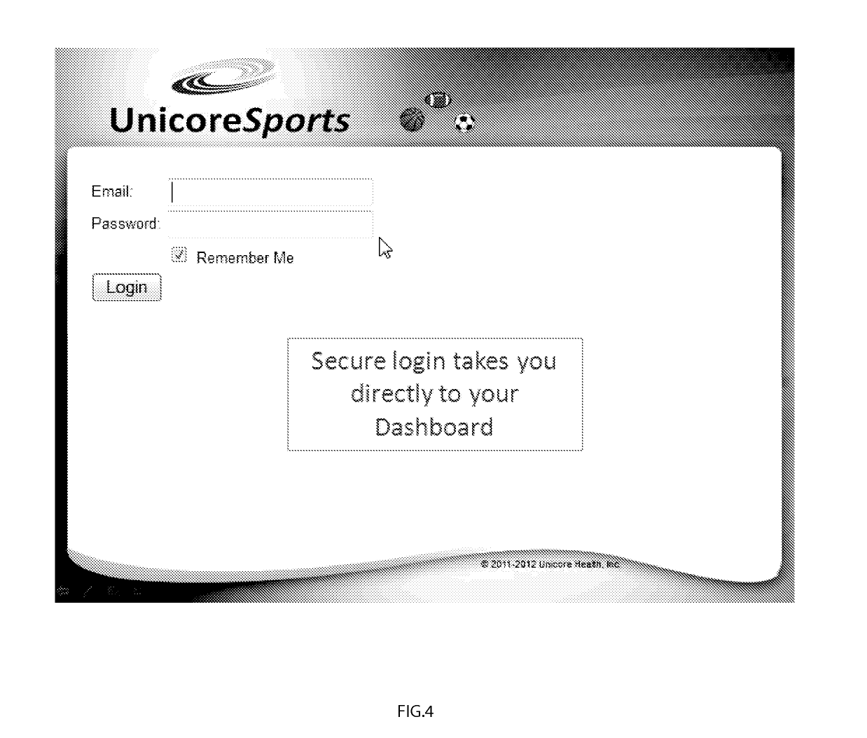

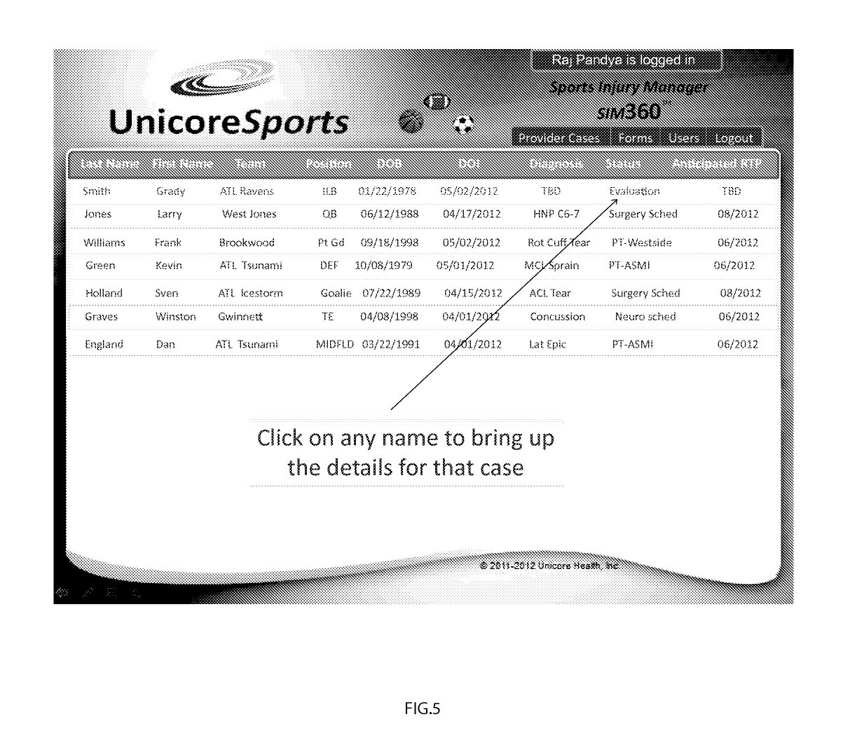

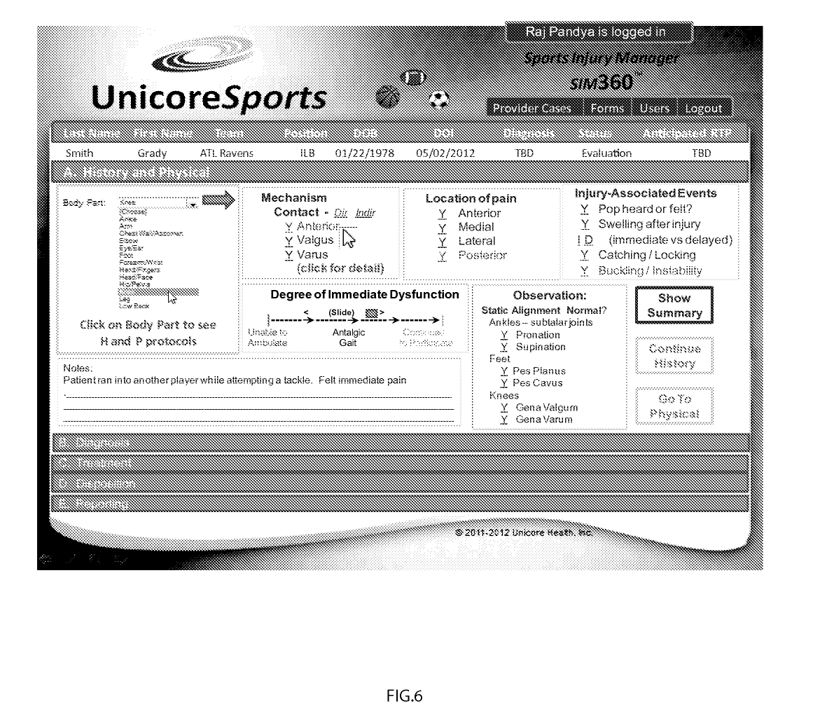

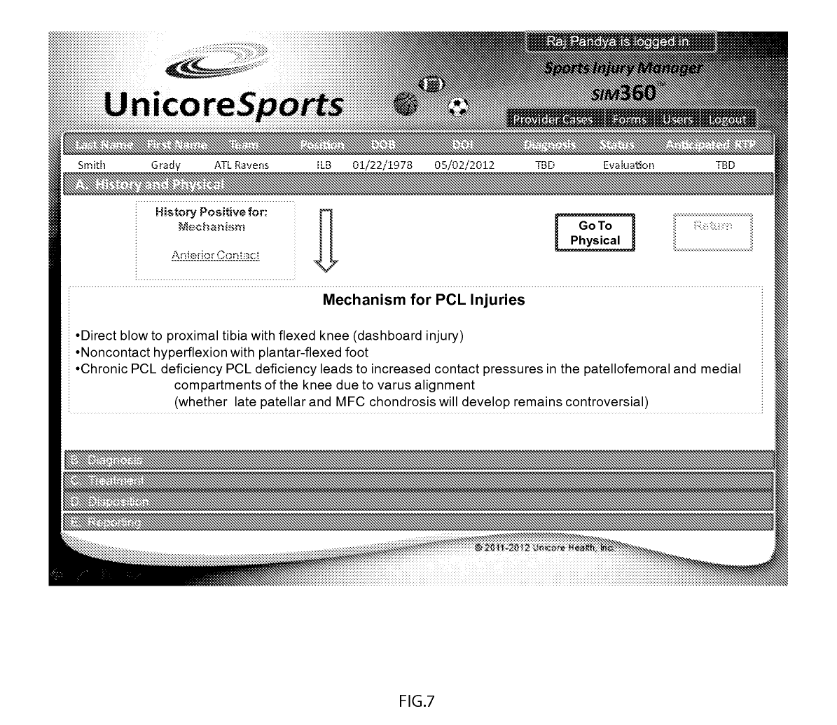

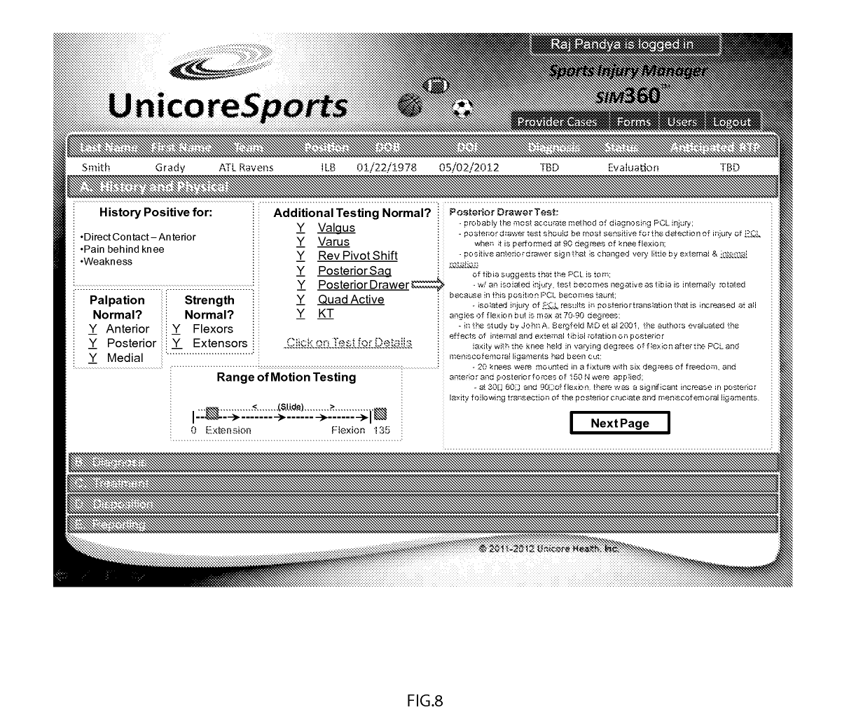

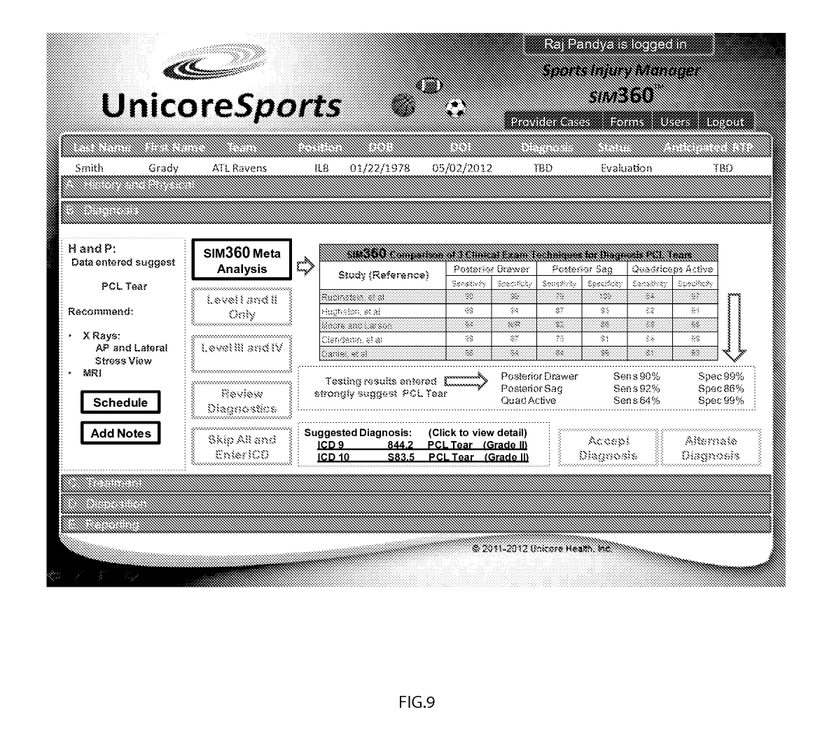

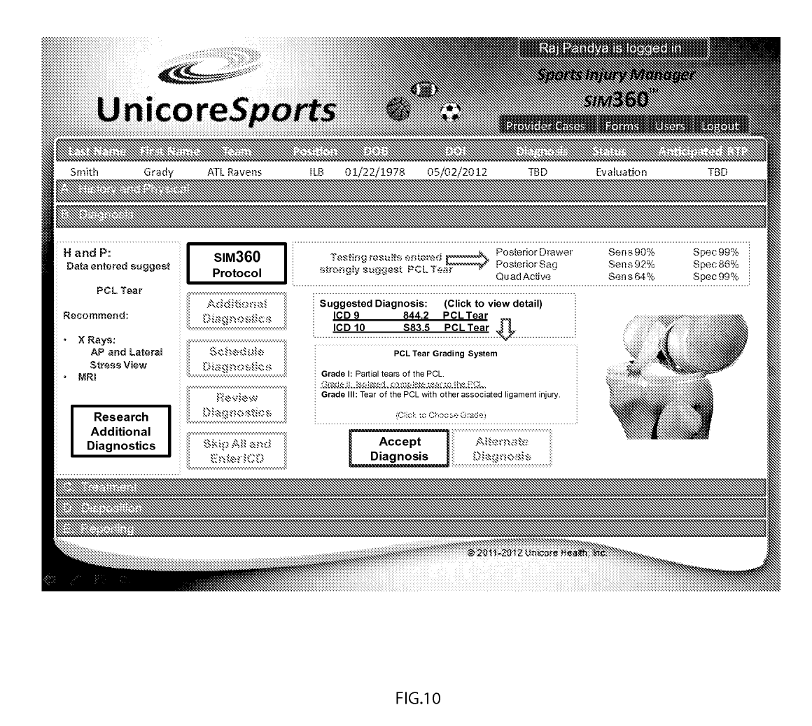

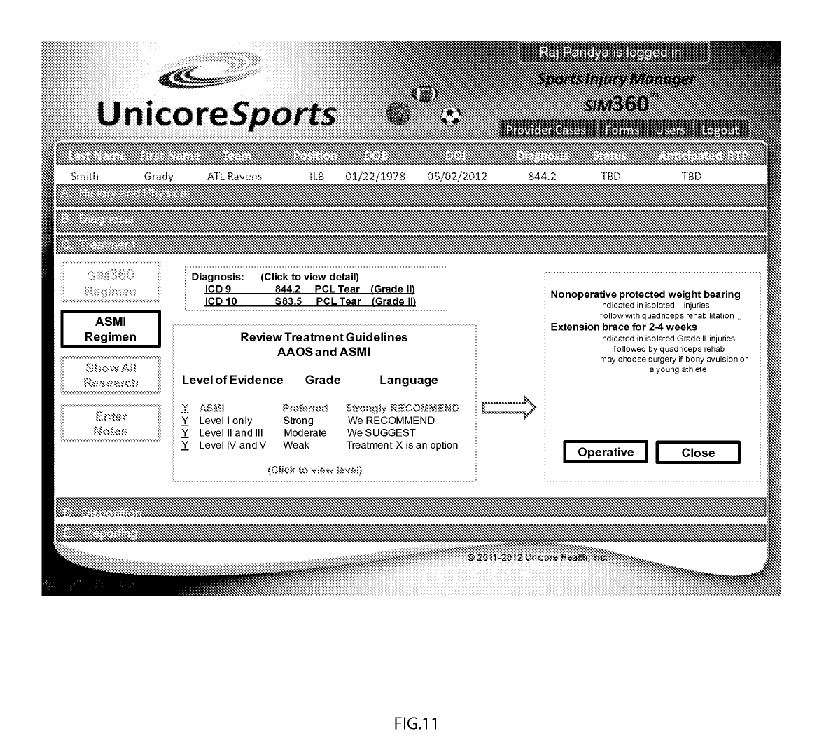

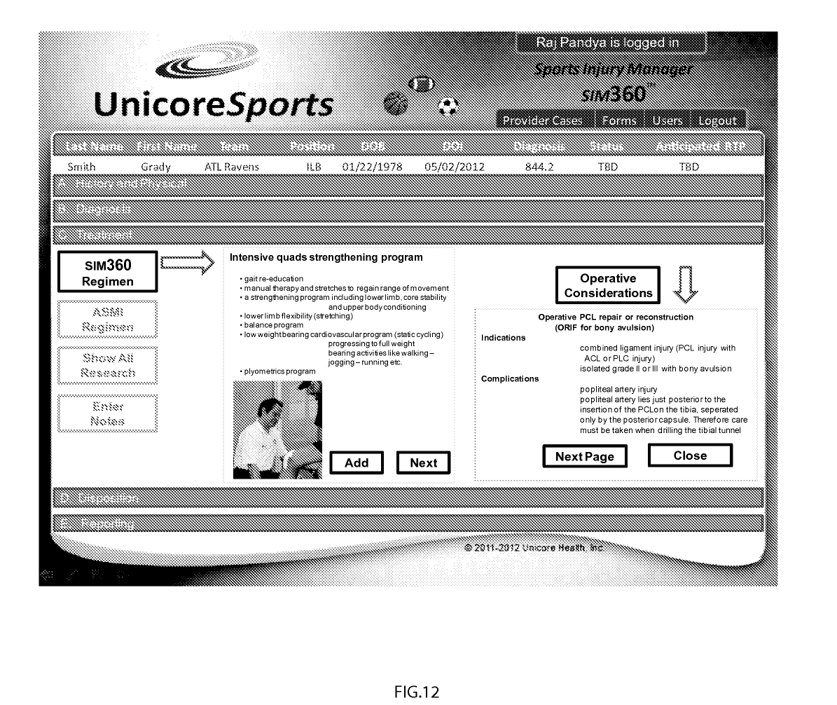

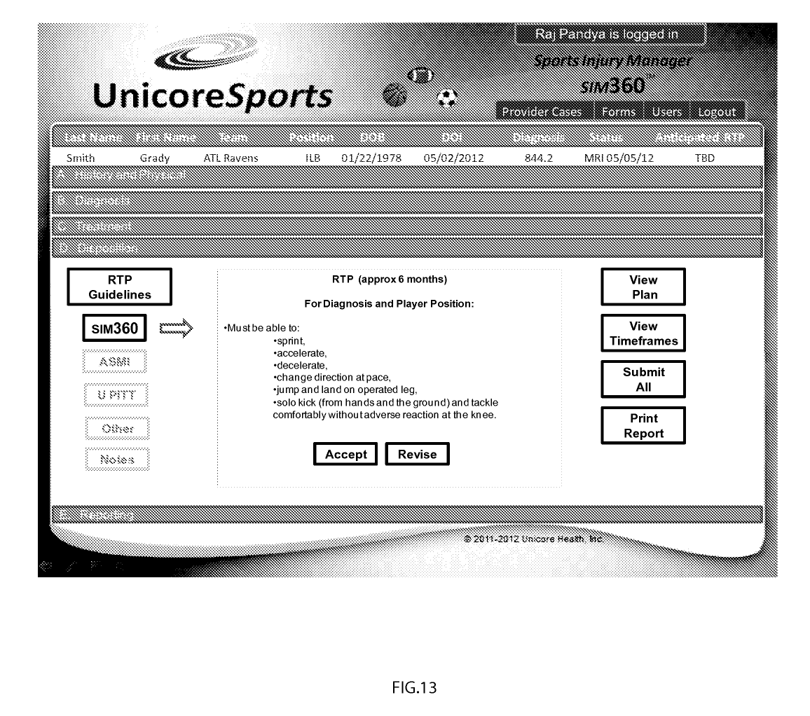

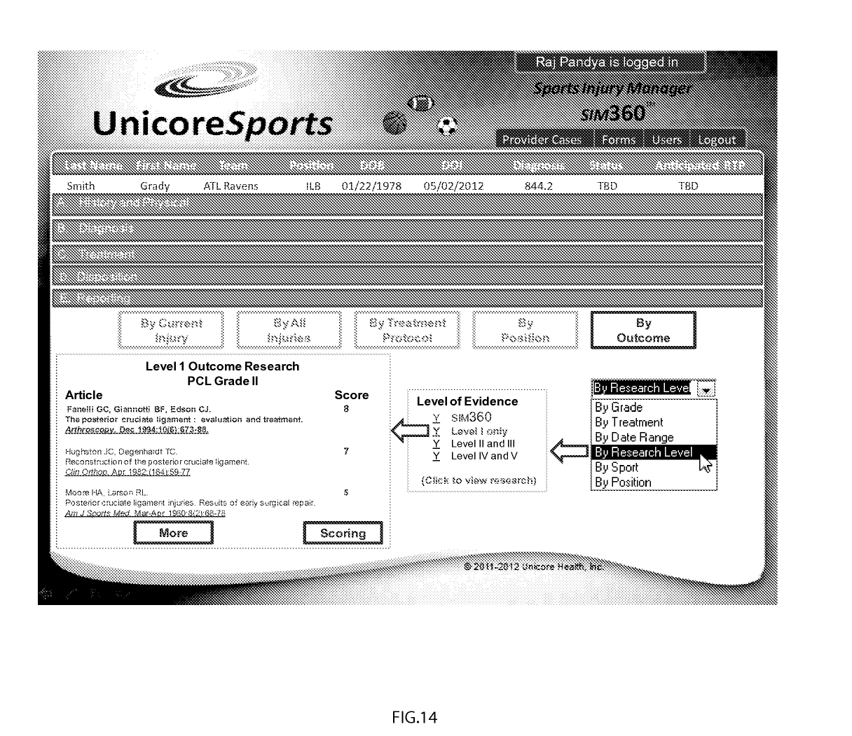

The ten screen shot FIGS. 4-14 show an exemplary use of the computer database. FIG. 4 shows that each user will have a secure login with password protection. Once logged in, the user sees FIG. 5 which is a dashboard listing each case showing the status and other case specific information. By selecting an injured player, the screen of FIG. 6 will open which shows the history and physical particulars i.e. mechanism, location, etc driven by the injured body part selected. By continuing on, more information is shown in FIG. 6. Continuing the program, FIG. 7 shows detail for mechanism component screen provides the user with definitions and criteria. FIG. 8 is an additional page for history and physical information still driven by body part. Selections on history and physical drive the next screen which narrows down diagnosis. FIG. 9 screen is research that shows the data summary for the tests conducted during the physical. It is a compilation of the different research with sensitivity and specificity—how accurate the tests are in differentiating a diagnosis. FIG. 10 is the summary of the diagnostic research as well as the testing completed by the physician during the exam. FIG. 11 shows the treatment regimen selected based on ASMI (Andrews Sports Medicine Institute) by level of research—were they controlled studies, anecdotal, etc. FIG. 12 is where the actual treatment is selected based on the criteria selected in the previous screen. It is diagnosis specific, and based on the scoring of outcome research. FIG. 13 is return to play (RTP) criteria, again driven by the choices selected in the previous screens. FIG. 14 is the reporting screen where reports can be viewed or submitted for scoring and review.

Additionally, after parsing the database, if no appropriate position is returned, then one option would be to change the medical treatment of the player to allow the player to be able to perform a different position. Using the alternative positions obtained from the database as a starting point, the physician can revise the player's treatment plan to allow the player to perform the alternative position. This new information can be inputted into the database for use in later situations, such as if a similar situation occurs, and the alternative position and treatment plan can be returned in the results of parsing the database.

The information gleaned and created during each of the above processes can be used by other utilities in determining whether the player can return to play in the same position, a different position, or not at all, as disclosed herein. This information also can be used by the physician and/or the team in prescribing whether a particular player can carry out a particular position or position task, or in modifying a particular position or position task, in that a particular player may no longer be able to carry out the position or a position task based on the medical diagnosis of the injury. Similarly, this information can be parsed when determining whether a particular position can be carried out by a particular player and in determining the risk of injury assessment of a particular position or position task, for current players, injured players, or new hires.

Thus it can be seen that the inventive database, utilities, and processes allow for a constant and continuous updating of a parsable database of criteria that are useful for various situations in determining whether a position is appropriate for a player and vice versa. This integrated parsable database comprises information on the team's positions, the team's players, injuries to a team's players, position candidates, required skills, required education, required certifications or credentials, governmental and legal requirements, and combinations of these. This database can be parsed according to predetermined criteria based on the utilities disclosed above so as to result in a match between a player and a position, between a diagnosis and a position, a possibility for adapting a player or a position to fit the criteria, and/or to assess the risk inherent in a position. As a result, this invention can facilitate return to play after an injury, can decrease lost player time and productivity by allowing a position to be modified or a player to return to play in a different, suitable position, and can provide for an elemental analysis of each position task and injuries related to each position task so as to assess the risk of injury of a position or individual position task.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A method for aiding a physician in examining, diagnosing and treating an injured patient comprising the steps of:
creating a computer database that converts a selected injured body part to a ICD code;
conducting an initial examination of an injured patient to identify the injury location or body part or multiple injury locations or body parts and inputting those locations or body parts into the computer database to identify an ICD code or codes;
creating an ICD examination diagnosis computer database to automatically convert an ICD code to a sorted group of one or more preliminary diagnoses with examination protocols for each preliminary diagnosis;
conducting recommended examination protocols and inputting examination results to the computer database to narrow diagnosis to most likely diagnosis;
selecting a recommended diagnosis;
creating a computer database of treatment protocols for every computer generated injury diagnosis, the treatment protocols having usage data and outcome data;
selecting a treatment protocol;
initiating treatment; and
inputting treatment outcome results back into the computer database for statistical inclusion into the computer database.

2. A method for determining whether an injured player/athlete can return to play in their current position, a different existing position, the current position but altered in some form, or a different existing position but altered in some form to result in lost time reduction, comprising the steps of:
(a) creating a computer database by conducting an elemental analysis of a player's position's functions and requirements for a particular sport by breaking a position down into elemental tasks and the physical and mental requirements of each task;
creating a parsable database of the positions, tasks and elements to establish specific maximum physical requirements and required movements; and
storing this elemental analysis on the computer database;
(b) conducting an initial medical diagnosis of the injured player by a physician examination of the player and inputting the computer database with the initial injury report to determine the physical capabilities and limitations of the player wherein the medical diagnosis comprises the steps of:
a doctor seeing the injured player patient;
the doctor, inputting the data from the examination into the computer database based on the injury;
the computer database automatically diagnosing the player based on the inputted data:
the computer database making recommendations regarding the player's ability to handle certain tasks;
the doctor reviewing the computer database recommendations and prescribing the computer database recommendations as maximum allowable restrictions on activities of the player wherein the recommendations include specific maximum allowable physical and mental requirements and movements; and
the physician's office inputting the maximum allowable restriction data into the computer database; and
(c) using a risk assessment utility that is used to parse the computer database of past injuries to determine what elements of a position are more prone to causing injuries, wherein said risk assessment utility is used to also adapt a position or position elements to compensate or reduce the risks;
(d) comparing by computer the elemental analysis and the medical diagnosis computer generated data to determine whether the player can function in a new assignment of a particular position, wherein the particular position is either the current position, the different existing position, the current position but altered in some form, or the different existing position but altered in some form, by performing the step of:
comparing the injured player's inputted computer generated medical diagnosis of allowable physical and mental requirements and movements with each position's elemental analysis requirements to either:
allow the injured player to safely return to play by first determining if the injured player's allowable capability exceeds each possible new assignment of elemental position requirements and then using the risk assessment utility to assess the risk for each position and altered position to assign a position for the injured player that minimizes the risk of injury for the injured player based on the computer database of past injuries; or
prevent the injured player from performing any position with tasks in the computer database exceeding the injured player's allowable restrictions on activities.

3. The method as claimed in claim 2, wherein:
(e) the computer database comprises information selected from the group consisting of the team's positions, the team's players, injuries to a team's players, position candidates, required skills, required education;
(f) the computer database is searched by computer according to predetermined search criteria so as to result in a match between a player and a position, between a diagnosis and a position, a possibility for adapting a player or a position to fit the criteria, or to assess the risk inherent in a position; and
(g) determining whether the player can do the position.

4. A computer system executing the steps for determining whether an injured player can return to play in their current position, a different existing position, the current position but altered in some form, or a different existing position but altered in some form, comprising:
(a) creating a computer database for storing and comparing software utilities;
(b) an elemental analysis utility for creating and using an integrated database of positions, position functions, position tasks, and position requirements for an team by breaking a position down into elemental tasks and the physical and mental requirements of each task;
creating a parsable database of the positions, tasks and elements to establish specific maximum physical requirements and required movements; and
storing this elemental analysis utility on the computer database;
(c) a medical diagnosis utility for creating and using an integrated database of player injuries, and of injuries to a particular player and storing the medical diagnosis utility and the integrated database of player injuries on the computer database;
wherein a doctor seeing an injured player, diagnoses the player by inputting the injury into the medical diagnosing utility, wherein the medical diagnosis utility then makes recommendations regarding the player's ability to handle certain tasks; wherein the doctor prescribes the computer generated recommendations as maximum allowable restrictions on activities of the player including maximum allowable physical and mental requirements and movements;
(d) a risk assessment utility for parsing the computer database of past player injuries to determine what elements of a position are more prone to causing injuries, wherein said risk assessment utility is used to also adapt a position or position elements to compensate or reduce the risks; and
(e) a return to play utility to determine whether an injured or disabled player can return to play in a new assignment of a particular position, wherein the particular position is either the current position, the different existing position, the current position but altered in some form or the different existing position but altered in some form, by performing the steps of:
comparing by computer the medical diagnosis of allowable physical and mental requirements and movements with each position's elemental analysis requirements to either:
allow the injured player to safely return to play by first determining if the injured player's allowable capability exceeds each possible new assignment of elemental position requirements and then using the risk assessment utility to assess the risk for each position and altered position to assign a position for the injured player that minimizes the risk of injury for the injured player based on the computer database of past injuries; or
prevent the injured player from performing any position with tasks in the computer database exceeding the injured player's allowable restrictions on activities.

5. The computer system for executing the steps as claimed in claim 4, wherein:
(f) the computer database comprises information selected from the group consisting of the team's positions, the team's players, injuries to a team's players, position candidates, required skills, required education;
(g) the computer database is searched by the computer according to predetermined search criteria so as to result in a match between a player and a position, between a diagnosis and a position, a possibility for adapting a player or a position to fit the criteria, or to assess the risk inherent in a position; and
(h) determining whether the player can do the position.
